(19) United States
(12) Patent Application Publication
(10) Pub. No.: US 2003/0213007 A1
(43) Pub. Date: Nov. 13, 2003

(54) Human milk produced by human mammary tissue implanted in non-human host animals and uses thereof

(76) Inventors: Charles Wilbur Slattery, Yucaipa, CA (US); Aladar Antal Szalay, Highland, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/402,247

(22) Filed: Mar. 26, 2003

Related U.S. Application Data
(60) Provisional application No. 60/368,631, filed on Mar. 27, 2002.

(51) Int. Cl. A01K 67/027

(52) U.S. Cl. 800/15; 800/14; 800/16

(57) ABSTRACT

The invention discloses chimeric milk-producing tissues containing human mammary cells implanted into cleared mammary fat pad tissue or other suitable tissue of a non-human animal host, and discloses the use of human milk produced by chimeric milk-producing tissues. The invention further provides methods for avoiding problems of xenogeneic transplantation in chimeric milk-producing tissues.

### CROSS-REFERENCE TO RELATED APPLICATION

This application claims priority to U.S. Provisional Application No. 60/368,631, filed Mar. 27, 2002, which is hereby incorporated by reference in its entirety.

### FIELD OF THE INVENTION

The present invention relates generally to production and use of milk from chimeric milk-producing tissues comprising human mammary tissue implanted in non-human host animals.

### BACKGROUND OF THE INVENTION

For healthy human mothers not exposed to contaminating environmental pollutants or toxins, mother's milk constitutes the best food for full-term, vigorous human infants. Unfortunately, most infants are not breast fed at all, or if they are breast fed it is not for an adequate period of time. In developed countries, it is often a matter of convenience. Occasionally, the mother may get sick and be required to take medicines that can be secreted into the milk and be harmful for the baby. There are special problems in areas of the world where HIV is prevalent, since this disease may be transmitted to the infant through the mother's milk. Present health recommendations are that, in most situations, infants should be breast fed for at least one year. Secondarily, a mother who cannot or does not wish to breast feed should be encouraged to pump her milk and provide her own milk to her child via a bottle. If neither of these options is feasible, it is recommended that a wet nurse be provided so that the child will get human milk. Feeding with infant formula should be the last option. Despite this, there is a huge business in infant formulas worldwide and a great deal of effort has gone into preparing infant formulas that are closer and closer to human milk in every aspect. The fact that these efforts are proceeding vigorously, with the continuous discovery of important new characteristics of human milk followed by attempts being made to modify infant formulas to match these characteristics, suggests that there is still a need for a better product to feed babies and promote optimal health. There are a number of important differences between human milk and infant formulas produced from the milk of other mammals or from soy or other proteins.

Overall Milk Composition

Milk produced by the human lactating mammary gland is very complex and different from that produced by most other mammals. For example, human milk is higher in fat and milk sugar (lactose) than cow's milk but lower in protein and mineral matter (See Packard, Human Milk and Infant Formula, Academic Press, New York, N.Y. (1982) the disclosure of which is herein incorporated by reference). Categorization of milk into a particular nutrient class based on its composition is regulated by governmental agencies. However, even within a nutrient class, there are important differences between milk produced by humans and milk produced by other mammals.

Fatty Acids

The fatty acid composition of the fat fraction of human milk may be very important for proper infant development. Certain short-chain fatty acids in cow's milk are not present in human milk and the ratio of saturated to unsaturated fatty acids is different. Because of this, many infant formulas are made with some vegetable oil to correct the discrepancy. This has also resulted in many proposals as to how to “humanize” the fats in infant formulas, for example as described in U.S. Pat. No. 6,034,130 to Wang, et al.

Non-Protein Nitrogen

There is a striking difference between the amount of non-protein nitrogen or NPN in human and cow's milk, human milk having about 5 times higher NPN. NPN is defined as all nitrogen-containing compounds not defined as protein which are no doubt a nutritional advantage to the infant. This would include peptides, free amino acids and various other organic and inorganic nitrogenous components. One of the free amino acids that may be important is taurine, which is the second highest amino acid in concentration in human milk but is almost absent in cow's milk. The relative abundance of other free amino acids also differs between human and cow's milk, and these differences may have an effect on the developing newborn. Since taurine is found in high levels in fetal brain tissues and also may be associated with cholesterol management in the body, it has been suggested that it be added to infant formula, e.g., U.S. Pat. No. 04,303,692 to Gaull.

Protein

The major protein fractions of milk are the caseins, which suspend calcium, phosphate, and other minerals needed for growth, and the whey proteins, which provide the remainder of the essential amino acids as well as providing other important functions. The composition of the casein and whey fractions is much different for humans than for other mammals. For example, human milk casein is mostly beta-casein and kappa-casein, containing very little alpha-casein (which is the major casein constituent of cow's milk) (Rasmussen et al., Comp. Biochem. Physiol., 111:75-81 (1995)). Furthermore, rather than having a fixed level of phosphorylation like other mammalian species, human milk beta-casein is phosphorylated at various levels from 0 to 5 (Groves and Gordon, Arch. Biochem. Biophys., 140:47-51 (1970)). Also, human milk kappa-casein is much more highly glycosylated than for other species (Dev et al., Preparative Biochemistry, 23:389-407 (1993)). The major whey proteins of human milk are alpha-lactalbumin and lactoferrin, whereas cow's milk whey is mostly beta-lactoglobulin. These differences have led to the suggestion that only the corresponding purified fraction from the cow's milk should be used in infant formulas since these most closely resemble the human proteins. Alternatively, recombinant human milk proteins produced in a variety of ways may be used exclusively or combined with some cow's milk proteins to make suitable formulas more closely resembling human milk, e.g., U.S. Pat. Nos. 5,795,611 and 5,942,274 to Slattery and 6,020,015 and 6,270,827 to Gaull.

Milk Fat Globule Membranes

Milks of the various mammals contain membrane material from the cells of the mammary gland. Milk fat globules are secreted from the lactating cell by envelopment in membranes which contain many proteins, some of which are highly glycosylated (See, Huston and Patton in Human Lactation, Jensen and Neville, eds., Plenum Publishing Corp., New York, N.Y. (1985); Mather, J. Dairy Sci., 83:203-247 (2000)). The glycosylation patterns in such proteins is unique to each species. The nutritional merits of these membranes is not entirely known but it is assumed that they may have a role in the proliferation of intestinal mucosal membrane of the neonate and certainly contribute some body-building materials upon digestion. However, more than thirty-five enzymes, proteins and other associated constituents of the milk fat globule membrane have been identified, many of which may be important to the newborn. It is certain that the constituents found in human milk fat globule membranes are not matched in any other mammal and it would be almost impossible to include all of these in infant formulas.

Various Nutritional and Protective Factors

Human milk contains a unique mix of hormones and related substances (see Human Lactation 3, Goldman et al, eds., Plenum Publishing Corp., New York, N.Y. (1987)) including thyroid hormones, human growth factors, nucleotides, prostaglandins, etc. Bile salt stimulated lipase is an enzyme found in the milk of humans and other primates but not in other mammals. Bile salt stimulated lipase aids the digestion of some fats and the recombinant form has been suggested as an additive to infant formula (See Tornell, U.S. Pat. No. 05,716,817). Lactoferrin is a protein in milk that has the ability to make iron unavailable and to interfere with the growth of disease bacteria in the gut. Depending on the stage of lactation, human milk contains from three to possibly one hundred times as much lactoferrin as cow's milk. Recombinant human lactoferrin has been suggested for use in many areas where resistance to bacterial infection is needed (see, for example, U.S. Pat. No. 6,111,081 to Conneely and Ward). Lysozyme is another antibacterial enzyme that is found in human milk at a much higher level than in cow's milk. Interferon is present in milk but only human interferon has the power to combat viral disease in humans.

Implantation of Mammary Tissue

In recent years, the “cleared” or epithelium-free mammary gland, having only the mammary fat pad, has been used to study mammary glands. Development and tumorigenesis in mammary glands has been investigated by reimplanting mammary tissue into cleared mammary glands, where some of the reimplanted mammary tissue may be cancerous (Medina, J. Mammary Gland Biology and Neoplasia, 1:5-19 (1996)). It has been found, for example, that implanted mammary cells will grow and eventually reproduce an entire functional gland (Kordon and Smith, Development, 125:1921-1930 (1998)). Recently, it has been shown that in ewes in which the mammary epithelium was completely excised and immediately replaced, the epithelium populated the mammary fat pad and synthesized milk that could be expressed from the teat (Hovey et al., J. Anim. Sci., 78:2177-2185 (2000)). Furthermore, mammary epithelial cells in culture may be genetically altered by gene introduction using retroviral vectors and then reimplanted into the mammary fat pad. These re-form an epithelium in which at least some cells express the introduced gene (Edwards et al., J. Mammary Gland Biol. Neoplasia, 1:75-89 (1996)). It is now recognized that the mammary fat pad is often used as a transplantation site (Neville et al., J. Mammary Gland Biol. Neoplasia, 3:109-116 (1998)). In fact, xenogeneic organs may even be placed into a mammary fat pad, which provides a good environment for growth and proper function, as disclosed in U.S. Pat. No. 5,434,341 to Outzen.

### SUMMARY OF THE INVENTION

The present invention provides chimeric milk-producing tissues in which human mammary cells, multipotent human stem cells or other human cells that may be induced to differentiate into mammary tissue, are placed into the cleared mammary fat pads or other suitable sites in non-human host animals capable of supporting development of human mammary tissue. The invention also includes human milk from chimeric milk-producing tissues. Preferably, the host animals are goats, cows, or sheep. In particular, the host animals are immature animals, where the mammary cells will be stimulated to grow and form functioning mammary glands in chimeric milk-producing tissues as the animals reach puberty, become pregnant and later begin lactating.

In accordance with another aspect of the invention, there is provided chimeric milk-producing tissue in which human mammary cells, multipotent human stem cells or other human cells that may be induced to differentiate into mammary tissue are placed into the cleared mammary fat pads or other suitable sites in transgenic host animals that have been engineered to express human major histocompatibility complex (MHC) antigens on their cells, such as fetal human leucocyte antigen, type G (HLA-G). In accordance with another aspect of the invention, the transplanted human cells may also be transformed so as to produce MHC antigens, such as HLA-G, conferring protection against rejection by the natural killer (NK) cells of the host.

In accordance with another aspect of the invention, there is provided chimeric milk-producing tissue in which the human mammary cells, multipotent human stem cells or other human cells that may be induced to differentiate into mammary tissue are immunoisolated from the cleared mammary fat pads or other suitable sites of host animals into which the human cells are introduced. Particular embodiments for immunoisolation include using biocompatible materials including encapsulation devices, membranes, and gels including hydrogels.

In yet another embodiment of the invention in which the hormones of the host animal are not as active on the human mammary cells as they might be on mammary cells of the host, recombinant human growth hormone will be administered to increase milk production.

In accordance with another aspect of the invention, there is provided human milk from host animals into which human mammary cells, multipotent human stem cells, or other human cells that may be induced to differentiate into mammary tissue have been implanted. In one embodiment, human milk from these animals will be collected and processed for storage or fed immediately to an infant as a replacement for mother's milk. In another embodiment, human milk will be collected from xenogeneic host cows producing human milk for use as infant food, where cows may be transgenic. In another embodiment, a large amount of milk is collected from host cows producing human milk, and is sterilized and packaged for bottle feeding of infants who might otherwise need to be fed with infant formula.

In accordance with another aspect of the invention, human milk produced in large quantities by host animals carrying functional human mammary cells may also become the source for the isolation of various human milk factors that may be used for different therapeutic purposes, including but not limited to lactoferrin, lactalbumin, beta-casein, kappa-casein, alpha-casein, and taurine. In various embodiments, human lactoferrin and human beta-casein can be used in combating bacterial infections. In other embodiments, human caseins may be isolated and formulated into pills or capsules for the delivery of calcium and other minerals to humans at all developmental stages to promote healthy bones and teeth, and in particular for delivery to the elderly, including those at risk for osteoporosis.

### DETAILED DESCRIPTION OF THE INVENTION

The present invention provides chimeric milk-producing tissues containing human mammary cells, multipotent human stem cells, or other human cells that may be induced to differentiate into mammary tissue, where these cells are placed into cleared mammary fat pads or other suitable sites in host animals. The present invention further provides human milk produced by chimeric milk-producing tissues. In one preferred embodiment, human mammary cells, multipotent human stem cells, or other human cells that may be induced to differentiate into mammary tissue, may be transplanted into cleared mammary fat pads or other suitable sites in immature host animals, such that the mammary cells will be stimulated to grow and form functioning mammary glands as the animals reach puberty, become pregnant and later begin lactating. Host animals of the present invention include (but are not limited to) cows, in particular Bos taurus, goats, sheep, other members of family Bovidae including other bovines, caprines, ovines, members of the family Cervidae including reindeer, members of the family Camelidae including camels, and any other mammal found to be suitable for the present invention.

To avoid incompatibility between introduced human cells and the host animal tissues, host animals may be transgenic animals that have been engineered to express human MHC antigens. Alternately, the human cells may be immunoisolated using, for example, membranes, hydrogels, or other encapsulation devices. The transplanted human-derived cells may also be transformed so as to produce MHC proteins, in particular HLA-G, to protect the human donor cells against rejection by the natural killer (NK) cells of the host.

Human milk from these animals can be collected and processed for storage or fed immediately to an infant as a replacement for mother's milk. In underdeveloped countries, a few goats, cows, or sheep carrying functional human mammary cells could supply all the human milk necessary in a village, in situations where mothers could not breast feed. For developed areas, large amounts of milk will be collected from transgenic/xenogeneic cows, sterilized and packaged for bottle feeding of infants who might otherwise need to be fed with infant formula. For purposes of clarification, “human milk” as used herein, including in the claims, refers to milk produced by human mammary cells in chimeric milk-producing tissue, where human milk of the present invention may include a small amount of non-human biological material from the host animal and/or may include a small amount of non-human genetic material, or a polypeptide encoded thereby, present in a genetic construct used for transformation of human cells or host animal cells.

Although some embodiments of the present invention utilize unmodified human mammary cells or precursors thereof implanted into cleared mammary fat pads of a non-human mammal, there may be some situations in which immunological intolerance of the implant could lead to rejection. Thus, one optional aspect of the present invention relates to various methods and techniques for reducing the immunological rejection of the implanted human cells.

Obtaining Viable Human Mammary Tissue Transplants in Non-Human Hosts

A. Induction of Specific Tolerance in the Host.

Embodiments of the present invention may utilize methods currently in use or under development to permit allogeneic (human to human) transplants to survive, the most popular being chronic immunosuppression. Embodiments of the present invention may utilize methods currently in use or under development to permit xenogeneic (cross-species) transplants as well, and many of these approaches are discussed by Brent (World J. Surg., 24:787-792 (2000)). Various approaches to allogeneic and/or xenogeneic transplantation can be adapted by one of skill in the art in order to obtain human milk suitable to feed to infants while excluding powerful immunosuppressive drugs from the milk.

A preferred approach is to develop a transgenic host animal that recognizes normal human mammary tissue as “self” and does not attack introduced human tissue(s). When using non-human hosts, a particularly preferred approach is to produce transgenic animals that express MHC antigens in the cells, including the fetal human MHC class I antigen HLA-G. This has been accomplished in mice transformed to express human MHC antigens (Schmidt and Orr, Immunol. Rev., 147:53-65 (1995)), where non-transgenic mice reject skin grafts from the transgenic mice as foreign, whereas allografts between transgenic mice were recognized as “self” (Schmidt et al., Human Immunol., 55:127-139 (1997)). Methods for producing transgenic animals are known in the art, for example the approach taught by DeBoer et al. (U.S. Pat. No. 5,633,076) for producing a transgenic bovine or any non-human mammal having a desired phenotype, or the teaching of Seebach et al. (U.S. Pat. No. 6,030,833) for production of transgenic animals, particularly swine, expressing human MHC antigens. One of skill in the art can use known methods of producing transgenic animals having a desired phenotype to produce transgenic animals that express HLA molecules, where the transgenic animals are suitable host animals for chimeric milk-producing tissues.

B. Production of Host Antigens in the Donor Tissue.

In one preferred embodiment, chimeric milk-producing tissues contain human donor cells that have been transformed to produce host antigens that protect the human cells from attack by the host animal's immunological defenses. Fetal tissue is protected from destruction by the mother through expression of a MHC class I antigen that prevents natural killer (NK) cell attack. In humans, this is HLA-G (Sasaki et al., Transplantation, 67:31-37 (1999)). Molecules with similar function are found in a number of primates and the gene sequences are known. Examples of primates having similar antigens are the rhesus monkey and the rhesus macaque (Mamu-AG) (Boyston et al., Immunogenetics, 49:86-98 (1999)). Similar fetal genes are found in the squirrel monkey (Sasc-G*02) (Cadavid et al., Proc. Natl. Acad. Sci. USA, 94:14536-14541 (1997), golden lion tamarin (Lero-G*01, GeneBank Accession No. (GB): B:U59643.1), brown-headed tamarin (Safu-G*01, GB:U59633.1), white-faced saki (Pipi-G*05, GB:U59656.1), white-tufted-ear marmoset (Caja-G*05, GB:U59641.1) and long-haired spider monkey (Atbe-G*03, GB:U59650.1). Genes encoding a similar protein are found in the mouse (Sipes et al., Immunogenetics, 45:108-120 (1996)) and possibly in the horse (Donaldson et al., Placenta, 15:123-135 (1994)).

Fetal MHC class I proteins have not been found for other species of interest, where there instead appears to be a down-regulation of transcription. The mRNA may be found in the trophoblast, but the animal may lack detectable fetal class I protein (Ellis et al., J. Reprod. Immunol., 37:103-115 (1998)). However, there are class I antigens expressed in the cow (bovine or BoLA) (Davies et al., Animals Genetics, 28:159-168 (1997)), the sheep (ovine or OLA) (Todd et al., J. Reprod. Immunol., 37:117-123 (1998)) and the goat (caprine or CLA) (Ruff et al., Rev. Elev. Med. Vet. Pays. Trop., 46:205-207 (1993)). Seebach et al. (U.S. Pat. No. 6,030,833) teach methods whereby such antigens may be tested for activity by determining if they can inhibit host NK cell mediated attack of target cells. Thus, one of skill in the art can prepare variants of the protein by various mutagenesis techniques until a variant is found that has the proper activity. Genes for the active antigens may then be prepared for insertion into the human donor cells with the desired result that such expression of active antigens will protect human cells against attack by host animal defenses such as NK cells. Although this should be successful in the production of human milk, the human cells of the chimeric milk-producing tissue would contain foreign genetic material and foreign protein. Although this foreign protein would be found in the milk from chimeric milk-producing tissues, along with the shed cells and cellular debris that is normally there, the milk is still considered human milk in accordance with the present invention. It is expected that the foreign protein would be present only in minor amounts and would not pose a problem.

C. Immunoisolation of Donor Cells

In an alternate preferred embodiment, immunoisolation of implanted human cells is employed to avoid the use or need for drugs to suppress immune responses and/or genetic engineering to induce immune compatibility. Immunoisolation in accordance with the present invention permits the introduction and maintenance of functional human mammary cells in a xenogeneic host by means of a bioartificial implant that is biocompatible with the host, such that donor-cell-containing implant does not provoke a foreign body response. A foreign body response may be caused by a material on the surface of the implant, antigens shed by the cells within, or by a combination of both, where the response can include fibrosis eventually leading to the formation of a capsule of connective tissue that isolates and starves the donor cells. The current state of knowledge is such that one of skill in the art can design a biocompatible immunoisolation device to permit introduction and maintenance of functional human mammary cells in a xenogeneic host. One approach involves the manufacture of an encapsulating membrane around viable cell cultures of human mammary cells, multipotent human stem cells, or other human cells that may be induced to differentiate into mammary tissue. For example, there are provided microcapsules or microspheres encapsulating a microscopic droplet of cell solution, where the microcapsule or microsphere are integral structures that do not require post-production sealing. In another embodiment, there are provided thin sheets which enclose cells, where the sheets are completely biocompatible over extended periods of time and do not induce fibrosis in the host, for example as disclosed in U.S. Pat. No. 6,125,225 to Antanavich et al. In accordance with aspects of the present invention, human mammary cell-containing thin sheets are provided which have dimensions allowing maintenance of optimal tissue viability through rapid diffusion of nutrients and oxygen and allowing changes in the milk secretion rate in response to changing physiology. Optionally, the mammary cell-containing sheets are easily retrievable if desired. In an alternate embodiment, human mammary cells may be immunoisolated using a sealed, implantable, encapsulation device for diffusing a biologically active product that crosses a selective membrane similar to that taught in U.S. Pat. No., 5,923,460 to Mills. Yet another embodiment utilizes polymers, hydrogel, or foam scaffolding in which human mammary cells capable of secreting milk are dispersed in cell-permissive pores in a biocompatible polymer, foam, or hydrogel jacket encapsulating the cell growth matrix, and the jacket has at least one selectively permeable membrane surface having a molecular weight cut-off which permits passage of milk substances across the membrane while being impermeable to cells, in an immunoisolation system similar to that disclosed in U.S. Pat. No. 6,054,142 to Li et al.

Another aspect of the present invention provides implantations made using these bioartificial implants, where implantation may occur in cleared mammary fat pads or other suitable sites. A further aspect of the invention provides human milk produced by chimeric milk-producing tissues containing immunoisolated human mammary cells in a host animal, and yet another aspect of the present invention is the chimeric host animals themselves.

D. Antigen Production in Both Host and Donor.

A preferred embodiment to prevent rejection of the human mammary cells or tissue by the non-human host is to produce transgenic host animals and transgenic human donor cells that both express similar human MHC antigens. For example, a transgenic host animal expressing HLA-G could be implanted with transformed human mammary cells expressing HLA-G under the control of a human promoter. This would minimize the amount of foreign genetic material and protein in the milk, although there could still be a very small amount of foreign genetic material because of the necessity for using a gene, perhaps from bacteria, most commonly as a selectable marker for transformation.

E. Tissue and Cells for Transplantation.

Human breast tissue is obtained from reduction mammoplasty and cut into thin slices from which the majority of adipose tissue is removed. These slices may be maintained in M199 holding media plus antibiotics for a few hours until implanted or frozen immediately in liquid N₂ for later use (Paul, Cell and Tissue Culture, 5^{th} Ed., Churchill Livingston, N.Y. (1975)). Primary cell cultures, derived from the human breast tissue obtained from reduction mammoplasty, are established by standard published procedures (Paul, Cell and Tissue Culture, 5^{th} Ed., Churchill Livingston, N.Y. (1975); Brooks, et al., Int. J. Cancer 73:690-696 (1997)) and are stored dry at −80° C. until preparation for implantation. They are then prepared for implantation by suspension in culture medium and grown until nearly confluent. The subconfluent cells are harvested and again suspended in medium after which they are centrifuged and washed with phosphate-buffered normal saline (PBS). These tissues and cell cultures will contain stem cells (Chepko and Smith, J. Mammary Gland Biology and Neoplasia 4:35-52 (1999)) that will regenerate mammary tissue upon implantation. A method to enhance the relative concentration of stem cells will enable transplantation with injection of fewer cells.

F. Surgical Procedure.

In one exemplary embodiment of the implantation procedure, the mammary tissue of a goat at 1-4 days of age is prepared for implantation by a modification of the procedure described for ewe lambs by Hovey, et al. (J. Anim. Sci. 78:2177-2185 (2000)). The animals are sedated and the mammary glands are locally anesthetized. An incision is made nearly circumscribing the base of each teat and subcutaneous blunt dissection is performed to beyond the bounds of the palpable parenchymal tissue and then through the extraneous adipose tissue of the mammary fat pad so that the adjacent parenchymal tissue can be completely removed. Parenchymal tissue is also removed from the area at the base of each teat up to the opening into the teat, whereupon tissue slices for implantation are placed into each fat pad to replace the parenchymal tissue just under each teat, the teats are immediately replaced into the excision sites and the sites are closed with wound clips. Alternately, the teats may be replaced without implanting tissue. Sterility is maintained by conventional means until the incisions are healed. If no tissue was implanted, cells from the cell culture suspension, optionally in PBS, may then be injected through the nipple and the teat cistern to the cleared fat pad at the base of the teat by means of a Hamilton syringe and a #28 needle.

Signs of rejection will be monitored closely. These include fever, malaise, pain or tenderness around the implant, fluid retention, a sudden increase in blood pressure, and a change in heart rhythm, urine color or smell, or bowel habits. These could occur immediately or up to 2 weeks after transplantation.

At 9-10 months of age, the animal may be impregnated, more generally from 10-15 months of age, and the development of the mammary tissue is then monitored by cell biology methods, e.g., with FISH (fluorescence in situ hybridization), involving immunocrossreaction and microscopic analysis, to determine if the tissue is truly of human origin. Postpartum milk production is similar to that of an unmodified animal. The milk produced is largely identical to native human milk, with only a tiny fraction of the non-human protein that would ordinarily be found in goat milk.

Although the foregoing procedure discusses the use of non-immunologically modified tissue and animals, it should be understood that the same surgical procedures may be used with transgenic animals and genetically-modified tissues in essentially any non-human mammal.

The patents and other references referred to in this disclosure are individually incorporated by this reference. Although the present invention has been described in connection with particular preferred embodiments, the full scope of the present invention is to be determined with reference to the literal scope of the claims that follow, together with all permissible equivalents thereof.

What is claimed is:

1. Chimeric milk-producing tissue comprising human donor cells comprising human mammary cells, multipotent human stem cells or other human cells that may be induced to differentiate into mammary tissue, wherein said human donor cells are placed into cleared mammary fat pads or other suitable sites in non-human host animals, wherein said cleared mammary fat pads or other suitable sites are capable of supporting development of human mammary tissue.

2. The chimeric milk-producing tissue of claim 1, wherein said non-human host animals are transgenic host animals expressing human major histocompatability complex (MHC) antigens.

3. The chimeric milk-producing tissue of claim 2, wherein said human MHC antigen is fetal human leucocyte antigen, type G (HLA-G).

4. The chimeric milk-producing tissue of claim 1, wherein said human donor cells are transgenic human donor cells expressing MHC antigens that protect said human donor cells from non-human host cell defenses.

5. The chimeric milk-producing tissue of claim 4, wherein said human MHC antigen is HLA-G.

6. The chimeric milk-producing tissue of claim 1, wherein said non-human host animals are transgenic host animals expressing human MHC antigens and said human donor cells are transgenic human donor cells expressing MHC antigens.

7. The chimeric milk-producing tissue of claim 1, wherein said human donor cells are immunoisolated.

8. The chimeric milk-producing tissue of any one of claims 1 to 7, wherein said host animals are immature, such that mammary cells will be stimulated to grow and form functioning mammary glands as said mammals reach puberty, become pregnant, and begin lactating.

9. The chimeric milk-producing tissue of any one of claims 1 to 7, wherein said non-human host animals comprise bovines, caprines, ovines, cervids, and camels.

10. The chimeric milk-producing tissue of any of claims 1 to 7, wherein said non-human host animals comprise bovines.

11. Human milk produced by the chimeric milk-producing tissue of claim 1.

12. Use of human milk produced by the chimeric milk-producing tissues of claim 1 as a source of nourishment for humans.

13. Human milk of claim 12, wherein said humans are infants.

14. Use of human milk produced by the chimeric milk-producing tissue of claim 1 as a source for isolation of human milk factors comprising lactoferrin, lactalbumin, beta-casein, kappa-casein, alpha-casein, and taurine.

15. Method of generating chimeric milk-producing tissues comprising the steps of:
a) obtaining human donor cells comprising human cells capable of differentiation into mammary tissue;
b) placing said human donor cells into cleared mammary fat pads or other suitable sites in non-human host animals, wherein said cleared mammary fat pads or other suitable sites are capable of supporting development of human mammary tissue;
c) measuring development of human mammary cells in chimeric tissue.

16. Method of producing human milk from chimeric milk-producing tissues of claim 1 comprising:
a) obtaining human donor cells comprising human cells that may be induced to differentiate into mammary tissue;
b) placing said human donor cells into cleared mammary fat pads or other suitable sites in non-human host animals, wherein said cleared mammary fat pads or other suitable sites are capable of supporting development of human mammary tissue;
c) exposing said non-human host animal to conditions suitable for milk production; and
d) obtaining milk from said non-human host animal having chimeric milk-producing tissues.

17. A non-human mammal having human-derived mammary cells inserted within cleared mammary fat pads of said mammal.

18. The mammal of claim 17, wherein said mammary cells produce human milk.
